(19) United States
(12) Patent Application Publication
(10) Pub. No.: US 2026/0240731 A1
(43) Pub. Date: Aug. 20, 2026

(54) COSMETIC COMPOSITION AND USES THEREOF

(71) Applicant: Coty Inc., New York, NY (US)

(72) Inventors: Ornella Frisina, MONACO (MC); Elisabeth Belin, MONACO (MC); Olivier Doucet, Roquebrune Cap Martin (FR)

(21) Appl. No.: 19/157,628

(22) PCT Filed: Feb. 16, 2024
(86) PCT No.: PCT/US2024/016156

§ 371 Date: Aug. 18, 2025

(30) Foreign Application Priority Data
Feb. 20, 2023 (FR) FR2301542

(51) Int. Cl. A61K 8/06; A61K 8/73; A61K 8/9728; A61K 8/99; A61Q 19/00; A61Q 19/08

(52) U.S. Cl. CPC A61K 8/062; A61K 8/73; A61K 8/9728; A61K 8/99; A61Q 19/007; A61Q 19/08; A61K 2800/48

(57) ABSTRACT

Various aspects disclosed relate to a cosmetic composition. The cosmetic composition includes water, an emulsifier, a thickener, and an active mixture. The active mixture includes kombucha, a mixture of Thermus thermophillus ferment and glycerin, and a-glucan oligosaccharide. A wt:wt ratio of the emulsifier to the thickener are in a range of from about 5:1 to about 1.5:1. A minimum stability of the cosmetic composition is measured at 8 weeks in a range of from about 30° C. to about 50° C. A pH of the cosmetic composition measured at a time of manufacturing and at 2 months following manufacturing is in a range of from about 5 to about 6. A viscosity of the cosmetic composition 24 hours after manufacture and at 2 months following manufacturing is in a range of from about 30,000 cps to about 80,000 cps.

### CLAIM OF PRIORITY

This patent application claims the benefit of priority to French Application Serial No. FR2301542, filed Feb. 20, 2023, which is incorporated by reference herein in its entirety.

### BACKGROUND

Cosmetic compositions that are topically applied are used for various applications. Desirable applications include reducing the appearance of wrinkles and/or moisturizing. It is therefore desirable to develop cosmetic compositions that can improve the ability to reduce wrinkles and/or moisturize skin.

### SUMMARY OF THE INVENTION

Various aspects disclosed relate to a cosmetic composition. The cosmetic composition includes water, an emulsifier, a thickener, and an active mixture. The active mixture includes kombucha, a mixture of Thermus thermophillus ferment and glycerin, and α-glucan oligosaccharide. A wt:wt ratio of the emulsifier to the thickener are in a range of from about 5:1 to about 1.5:1. A minimum stability of the cosmetic composition is measured at 8 weeks in a range of from about 30° C. to about 50° C. A pH of the cosmetic composition measured at a time of manufacturing and at 2 months following manufacturing is in a range of from about 5 to about 6. A viscosity of the cosmetic composition 24 hours after manufacture and at 2 months following manufacturing is in a range of from about 30,000 cps to about 80,000 cps.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %

The present disclosure relates to cosmetic or dermopharmaceutical compositions containing, for the care of the skin, mucosae and skin appendages, and in some examples for preventing the signs of endogenous and/or exogenous ageing.

The skin, mucous membranes, and the skin appendages are fragile elements which deserve the greatest regards. Indeed, their balance is disturbed permanently by external aggressions, sun, wind, cold. The skin ageing is one of the first external signs of ageing. It results from a certain number of deteriorations which occur spontaneously in the course of the time but which can also be induced by external factors (solar radiations, tobacco smoke, excessive alcohol consumption).

The signs of ageing result clinically by the appearance of wrinkles and small lines, in a slackening of the cutaneous and subcutaneous tissues which result in a dull texture of skin, a slackening of the cutaneous microrelief, a decreased cutaneous firmness, a loss of elasticity, and an overall flask, duller skin and without freshness of the complexion.

Ageing also results in a reduction in the number, thickness and growth of the hair, a fall of secretions (sebaceous and sweat), and a deterioration of the structure and growth of the nails. On the parts of the skin which were exposed to the sun throughout the life—primarily the face, the low neckline, hands and before arm—one often observes spots of hyper- or hypo-pigmentation.

Some of these signs are more particularly related to intrinsic or physiological ageing, i.e. with ageing related to the age, whereas others are more specific of extrinsic ageing, i.e. ageing caused generally by the environment; it is more particularly about photo-ageing due to the exposure to the sun, the light or any other radiation.

Various applications described herein relate to a cosmetic composition that can be used as a topical skin composition. For example, the cosmetic composition can be a facial cream. Even more specifically, the cosmetic composition can be used as a cream applied to skin proximate to a user's eyes. The cosmetic composition can reduce the appearance of wrinkles in the area of skin to which it is applied.

The cosmetic composition is in the form of an emulsion. The emulsion typically is an oil-in-water emulsion. In addition to the cosmetic composition's ability to reduce the appearance of wrinkles, the cosmetic composition has additional beneficial properties. For example, the cosmetic composition shows commercially acceptable stability. The cosmetic composition is considered to be stable if it does not phase separate. For example, the cosmetic composition shows that it is stable for a minimum time of 8 weeks after continuous exposure to temperatures ranging from about 30° C. to about 50° C., about 37° C. to about 45° C., less than, equal to, or greater than about 30° C., 31, 32, 33, 34, 35, 36, 37, 38, 39, 40, 41, 42, 43, 44, 45, 46, 47, 48, 49, or about 50° C. Thus, the cosmetic composition is able to be stored for a sustained period of time and be usable. Achieving the disclosed stability can be linked to ratios of the thickener to stabilizer as described herein.

The cosmetic composition additionally is able to maintain an acceptable pH. For example, when measured at least 2 months following manufacture, a pH of the composition can be in a range of from about 5 to about 6, about 5.1 to about 5.7, less than, equal to, or greater than about 5, 5.1, 5.2, 5.3, 5.4, 5.5, 5.6, 5.7, 5.8, 5.9, or about 6. The cosmetic composition's pH can dramatically affect its performance and stability. Therefore, the cosmetic composition's ability to maintain an acceptable pH over a sufficient amount of time enhances its ability to be a commercially viable product. The pH ranges described herein help to allow for the disclosed thickener to stabilizer ratios to be present.

Additionally, the cosmetic composition's viscosity is able to be controlled and remain in a suitable range. For example, a viscosity of the cosmetic composition measured at both 24 hours after manufacture and at 2 months after manufacturing is in a range of from about 30,000 cps to about 80,000 cps, 40,000 cps to about 70,000 cps, less than, equal to, or greater than about 30,000 cps, 35,000, 40,000, 45,000, 50,000, 55,000, 60,000, 65,000, 70,000, 75,000, or about 80,000 cps. The viscosity is measured with a Brookfield RVT E/20 at a temperature of 25° C. The viscosity of the cosmetic composition is such that the composition is spreadable but does not run or disperse too quickly. Additionally, the confirmation that the viscosity remains in the aforementioned range after 2 months demonstrates that the viscosity is stable and doesn't go above or below the range quickly. The viscosity can be controlled in part by the disclosed thickener to stabilizer ratios herein.

The aforementioned properties result from the constituents of the cosmetic composition. While not so limited the cosmetic composition includes water, an emulsifier, a thickener, and an active mixture. The emulsifier can include any number of components that are capable of forming the water-in-oil composition. A particularly suitable emulsifier is PEG-40 stearate. PEG-40 stearate is a synthetic polymer composed of PEG (polyethylene glycol) and stearic acid, a naturally occurring fatty acid. It can also be referred to as MACROGOL STEARATE 2000, POLYETHYLENE GLYCOL 2000 MONOSTEARATE, POLYOXYETHYLENE (40) MONOSTEARATE, POLYOXYL 40 STEARATE, and STEARETHATE 40.

The thickener can include any number of suitable thickeners to help achieve the desired viscosity values. For example, the thickener can include a mixture of sodium acrylates copolymer and lecithin. Sodium acrylate copolymer is a sodium salt of a polymer consisting of acrylic acid, methacrylic acid or one of their simple esters. Acrylates are a family of polymers which are a type of vinyl polymer. Synthetic chemicals used in the manufacture of plastics, paint formulations and other products. Acrylate copolymer is a general term for copolymers of two or more monomers consisting of acrylic acid, methacrylic acid or one of their simple esters. Sodium acrylate copolymer is also known as Acrylamide/sodium acrylate copolymer; 2-propenamide, polymer with 2-propenoic acid, sodium salt; 2-propenoic acid, sodium salt, polymer with 2-propenamide; 2-Propenamide-sodium 2 propenoate copolymer; sodium acrylate acrylamide polymer; sodium acrylate-acrylamide copolymer. Lecithin generally can include a mixture of glycerophospholipids including phosphatidylcholine, phosphatidylethanolamine, phosphati dylinositol, phosphatidylserine, and phosphatidic acid. Lecithin can be derived from soy bean. Soy bean derived lecithin can include about 33 wt % to about 35 wt % soybean oil, about 20 wt % to about 21 wt % phosphatidylinositols, about 19 wt % to about 21 wt % phosphatidylcholine, about 8 wt % to about 20 wt % phosphatidylethanolamine, about 5 wt % to about 11 wt % other phosphatides, about 5 wt % free carbohydrates, about 2 wt % to about 5 wt % sterols, and about 1 wt % moisture.

The emulsifier is present in the oil phase of the emulsion and the thickener is in the water phase of the emulsion. Relative to each other, a wt:wt ratio of the emulsifier to the thickener are in a range of from about 5:1 to about 1.5:1, about 5:4 to about 5:2, or about 5:3 to about 5:2.

The active mixture includes a mixture of components that act to together on the user's skin. Specifically, the active mixture can function to reduce the appearance of wrinkles, hydrolyze the skin, or the like. As an example, the active mixture can include kombucha; a mixture of Thermus thermophillus ferment and glycerin; and α-glucan oligosaccharide. Kombucha (also tea mushroom, tea fungus, or Manchurian mushroom; Latin name Medusomyces gisevii) is a fermented, lightly effervescent, sweetened black tea drink. Thermus thermophilus ferment is the natural result from the fermentation of Thermus thermophilus, a bacterium that can live thousands of feet below the ocean's surface. On skin, this special ferment can help protect skin's support structure from falling prey to destructive enzymes and help maintain a firm feel. It also has antioxidant ability, particularly on rogue molecules formed as fatty acids break down. The radical-scavenging ability of Thermus thermophilus has been compared to potent antioxidants in skin such as superoxide dismutase. It's a very good source of alpha-galactosidase, an enzyme which helps replenish glycoproteins, which skin needs to look healthier. α-glucan oligosaccharide is a natural prebiotic that helps support a healthy microbiome. When skin's microbiome (it's invisible barrier) is disrupted, the population of harmful bacteria can grow since it's not being kept in check by healthy flora on the surface. This has the potential to trigger signs of irritation and sensitized skin. α-glucan oligosaccharide also has antioxidant benefit, helping to reduce the visible effects of oxidative stress on skin.

Kombucha is in a range of from about 1 wt % to about 5 wt % of the cosmetic composition, about 2 wt % to about 4 wt %, less than, equal to, or greater than about 1 wt %, 1.5, 2, 2.5, 3, 3.5, 4, 4.5, or about 5 wt %. The mixture of Thermus thermophillus ferment and glycerin can be in a range of from about 1 wt % to about 10 wt % of the cosmetic composition, about 3 wt % to about 7 wt %, less than, equal to, or greater than about 1 wt %, 1.5, 2, 2.5, 3, 3.5, 4, 4.5, 5, 5.5, 6, 6.5, 7, 7.5, 8, 8.5, 9, 9.5, or about 10 wt %. α-glucan oligosaccharide can be in a range of from about 0.1 wt % to about 5 wt % of the cosmetic composition, about 0.5 wt % to about 3 wt %, less than, equal to, or greater than about 0.1 wt %, 0.5, 1, 1.5, 2, 2.5, 3, 3.5, 4, 4.5, or about 5 wt %. In total, the active mixture can be in a range of from about 1 wt % to about 15 wt % of the cosmetic composition, about 5 wt % to about 10 wt %, less than, equal to, or greater than about 1 wt %, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 wt %. The active mixture is located in the water phase.

The water phase and the oil phase can include other constitutes. For example, the oil phase can further include shea butter, pentaerythrityl tetraethylhexanoate, triglyceride, behenyl alcohol, glyceryl stearate, or a mixture thereof. The water phase further include glycerine, xanthan gum, or a mixture thereof. When present, shea butter can be in a range of from about 1 wt % to about 10 wt % of the cosmetic composition, about 3 wt % to about 7 wt %, less than, equal to, or greater than about 1 wt %, 2, 3, 4, 5, 6, 7, 8, 9, or about 10 wt %. When present, pentaerythrityl tetraethylhexanoateis in a range of from about 1 wt % to about 8 wt % of the cosmetic composition, about 2 wt % to about 5 wt %, less than, equal to, or greater than about 1 wt %, 2, 3, 4, 5, 6, 7, or about 8 wt %. When present, triglyceride is in a range of from about 1 wt % to about 10 wt % of the cosmetic composition, about 2 wt % to about 8 wt %, less than, equal to, or greater than about 1 wt %, 2, 3, 4, 5, 6, 7, 8, 9, or about 10 wt %. When present behenyl alcohol can be in a range of from about 1 wt % to about 5 wt % of the cosmetic composition, about 2 wt % to about 4 wt %, less than, equal to, or greater than about 1 wt %, 2, 3, 4, or about 5 wt %. When present, glyceryl stearate can be in a range of from about 1 wt % to about 5 wt % of the cosmetic composition, about 2 wt % to about 4 wt %, less than, equal to, or greater than about 1 wt %, 2, 3, 4, or about 5 wt %. When present glycerine can be in a range of from about 1 wt % to about 10 wt % of the cosmetic composition, about 3 wt % to about 7 wt %, less than, equal to, or greater than about 1 wt %, 2, 3, 4, 5, 6, 7, 8, 9, or about 10 wt %. When present xanthan gum can be in a range of from about 0.1 wt % to about 5 wt % of the cosmetic composition, about 0.2 wt % to about 0.7 wt %, less than, equal to, or greater than about 0.1 wt %, 0.5, 1, 1.5, 2, 2.5, 3, 3.5, 4, 4.5, or about 5 wt %.

A specific example of a cosmetic formulation according to the instant disclosure is provided below in Table 1. Table 1 provides the concentration (wt %), INCI name, and CAS no. (where available) for each component.

TABLE 1

| Wt % | INCI Name | CAS No. |
|---|---|---|
| 46-50 | Aqua/Water/Eau | 7732-18-5 |
| 4-8 | Propylene Glycol | 68583-51-7 |
| | Dicaprylate/Dicaprate | |
| 3-7 | Caprylic/Capric Triglyceride | 73398-61-5 |
| 3-7 | Glycerin | 56-81-5 |
| 3-7 | Butyrospermum Parkii (Shea) | 91080-23-8 |
| | Butter | |
| 2-6 | Pentaerythrityl | 7299-99-2 |
| | Tetraethylhexanoate | |
| 1-5 | Glyceryl Stearate SE | 11099-07-3 |
| 1-4 | Euglena Gracilis Extract | 100085-62-9 |
| 1-4 | Thermus Thermophillus | 100085-62-9 |
| | Ferment | |
| 1-4 | Saccharomyces/Xylinum/Black | 100085-62-9 |
| | Tea Ferment | |
| 1-4 | Behenyl Alcohol | 661-19-8 |
| 1-4 | PEG-40 Stearate | 9004-99-3 |
| 1-4 | Hydrogenated Polyisobutene | 68937-10-0 |
| 0.5-2 | Sodium Acrylates Copolymer | 25549-84-2 |
| 0.5-1.5 | Phyllostachys Bambusoides | 91771-33-4 |
| | Extract | |
| 0.4-1 | Perfum/Fragrance | — |
| 0.3-1 | Alpha-Glucan Oligosaccharide | — |
| 0.3-1 | Hydroxyacetophenone | 99-93-4 |
| 0.3-1 | Avena Sativa (Oat) Kernel | 84012-26-0 |
| | Extract | |
| 0.3-1 | Lecithin | 8002-43-5 |
| 0.1-0.5 | Butylene Glycol | 107-88-0 |
| 0.1-0.5 | Xanthan Gum | 11138-66-2 |
| 0.1-0.4 | Pseudoalteromonas Ferment | — |
| | Extract | |
| 0.1-0.4 | Chlorphenesin | 104-29-0 |
| 0.1-0.4 | Hydrolyzed Hyaluronic Acid | — |
| 0.05-0.2 | Citric Acid | 77-92-9 |
| 0.05-0.2 | Sodium Hyaluronate | 9067-32-7 |
| 0.03-0.08 | Potassium Sorbate | 24634-61-5 |
| 0.02-0.06 | Adenosine | 58-61-7 |
| 0.01-0.05 | Sodium Benzoate | 532-32-1 |
| 0.004-0.009 | Phellodendron Amurense Bark | 164288-52-2 |
| | Extract | |
| 0.003-0.007 | Caprylyl Glycol | 1117-86-8 |
| 0.0005-0.003 | Tocopherol | 10191-41-0 |

The cosmetic compositions described herein can be produced according to many suitable methods. For example, the water, emulsifier, thickener, and active mixture can simply be mixed together for period of time to form the cosmetic composition.

The cosmetic composition can be applied in any suitable manner. For example the cosmetic composition can be topically applied to a user's skin. The cosmetic composition can be topically applied by hand, with an applicator, or both. Examples of applicators can include brushes, pads, sponges, or the like.

### EXAMPLES

Various embodiments of the present invention can be better understood by reference to the following Examples which are offered by way of illustration. The present invention is not limited to the Examples given herein.

### Friction

A friction of the composition described herein will be determined with a texturometer. The texturometer will be a LS1 machine available from Ametek, Berwyn, PA. 15 ml of the composition will be loaded on a sample tray of the texturometer. A spatula will be used to form an even layer of the composition. A metal weight attached to a wire will be placed at the furthest end of the tray. The texturometer will pull the wire to cause the metal weight to be dragged across the surface of the composition. The texturometer will record the force required to move the weight and use that measured force to determine the friction of the composition.

It is expected that a majority of participants evaluating the composition will find that the composition feels comfortable when applied to the skin. It is further expected that the friction of the composition will allow the composition to not run to an undesirable degree following application.

### Penetration and Pick-Up

A penetration and pick-up assessment of the composition described herein will be determined with a texturometer. The texturometer will be a LS1 machine available from Ametek, Berwyn, PA. To determine these values, a sample cup will be filled with 100 ml of the composition. A spatula will be used to spread the composition and form an even layer. A probe of the texturometer will descend into the composition. The force to descend into the composition will be measured and that force will be the penetration value. Following descending, the probe will ascend and the force required will be measured as the pick-up value. The higher the load (Force) required to penetrate the sample the stronger the structure of the sample and therefore the thicker the sample will feel (the more it resists being penetrated) The higher the load required to remove the probe from the sample, the higher adherence a user will feel.

It is expected that a majority of participants evaluating the composition will find that the composition feels comfortable when applied to the skin. It is further expected that the penetration and pick-up of the composition will allow the composition to not run to an undesirable degree following application.

### Spreadability

A spreadability of the composition described herein will be determined with a texturometer. The texturometer will be a LS1 machine available from Ametek, Berwyn, PA. 50 ml of the composition is placed in a sample cup. A spatula will be used to spread the composition into an even layer. A cone probe will descend into the composition and the force to spread the product against the walls of the cone probe will be recorded to measure the spreadability of the composition. The higher the energy required to extend the machine the stronger the structure of the composition and therefore the lower the spreading of the composition.

It is expected that a majority of participants evaluating the composition will find that the composition feels comfortable when applied to the skin. It is further expected that the measured spreadability of the composition will allow the composition to not run to an undesirable degree following application.

### Other Sensory Attributes

A panel of consumers will be asked to evaluate several characteristics of the composition before, during, and after application. Table 1 provides the category (e.g., appearance, texture, odor) that will be evaluated, along with its definition, protocol and the scale panelists will be asked to use for evaluation. It is expected that the compositions tested will have acceptable scores from a majority of the panelists.

TABLE 1

| Category | | Descriptor | Definition | Protocol | Scale |
|---|---|---|---|---|---|
| Pick-up | Appearance | Glossiness | Quantity of light | Assess the gloss of | Mat-Glossy |
| (before | | | reflected by the | product by looking at the | |
| application): | | | product | thumb under white light | |
| 100 μl | Texture | Firmness | Strength required | Compress softly the | Low-High |
| of product | | | to compress the | product between the | |
| dropped off | | | product | thumb and the forefinger | |
| the thumb | Texture | Stickiness | Strength required | Separate softly the | Low-High |
| of each | | | to remove the | product between the | |
| panelist | | | forefinger from the | thumb and the forefinger | |
| | | | thumb | | |
| | Texture | Stringiness | Quantity of product | Compress the product | Low-High |
| | | | being stretched | between the thumb and | |
| | | | rather than broken | forefinger and separate | |
| | | | when separating | the fingers once. Make | |
| | | | the fingers | sure that the distance | |
| | | | | between the fingers is | |
| | | | | equivalent to one finger. | |
| During | Odor | Overall | Evaluate the overall | Bring forearm close to | Low-High |
| application: | | Fragrance | fragrance intensity | the nose and evaluate | |
| 50 μl of | | Intensity | on skin during | fragrance intensity | |
| product is | | during | application | | |
| dropped off | | application | | | |
| the forearm | Texture | Wetness | Quantity of water | After 3 massages | Low-High |
| of the | | | perceived during | | |
| panelist, at | | | application | | |
| the centre | Texture | Spreadability | Easiness to spread | After 3 massages | Low-High |
| of a 5*5 cm | | | the product on the | | |
| zone | | | skin | | |
| marked by | Texture | Thickness | Quantity of | After 12 massages | Low-High |
| the panel | | | products perceived | | |
| leader | | | between the skin | | |
| Each | | | and the fingers | | |
| panelist | Texture | Greasiness | Quantity of grease | After 15-20 massages | Low-High |
| spreads | | | perceived in the | | |
| the product | | | product during | | |
| within the | | | application | | |
| zone with 2 | Texture | Oiliness | Quantity of oil | After 15-20 massages | Low-High |
| fingers | | | perceived in the | | |
| (forefinger | | | product during | | |
| and middle | | | application | | |
| finger), | Texture | Lifting | Sensation of | On the back of the hand, | Low-High |
| doing | | | tightening pores, as | perform 15-20 | |
| circular | | | if the skin is | massages and assess | |
| movements | | | stretched | the feeling of tight skin, | |
| at a speed | | | | tightening pores, lifting | |
| of 2 | | | | effect 2 minutes after the | |
| turns/sec | | | | massages | |
| | Appearance | Noodling | Presence of little | After 15-20 massages | Absence-Presence |
| | | | flakes/balls/clumps | | |
| | | | on top of the skin | | |
| | | | during application | | |
| | Texture | Playtime | Time needed for | Count number of | Low-High |
| | | | the product to be | massages until the | |
| | | | fully rubbed in | product losses its | |
| | | | | humidity and hydration | |
| | | | | effect | |
| After | Appearance | Glossiness | Quantity of light | Assess the gloss of | Mat-Glossy |
| application: | | | reflected by the | product by looking at the | |
| After the | | | product | forearm under white light | |
| product | Texture | Stickiness | Strength required | Tap gently onto the skin | Low-High |
| fully rubbed | | | to remove the | where product was | |
| in, the | | | fingers from the | rubbed in, with forefinger | |
| panelist | | | skin | and middle finger | |
| wipes his | Texture | Sliding | Easiness to slide | Stroke the zone of the | Low-High |
| fingers and | | effect | fingers on the skin | skin where product was | |
| starts the | | | | rubbed in | |
| evaluation | Texture | Residue | Quantity of product | Stroke the zone of the | Low-High |
| | | | residue left on skin | skin where product was | |
| | | | after application | rubbed in | |
| | Texture | Residue | Evaluate the type | Stroke the zone of the | Greasy, |
| | | Type | of residue left on | skin where product was | Oily, |
| | | | skin after | rubbed in | Powdery, |
| | | | application | | Waxy, |
| | | | | | Chalky |
| | Texture | Velvety | Evaluate the velvet | Stroke the zone of the | Low-High |
| | | | feeling left on skin | skin where product was | |
| | | | after application | rubbed in | |
| | | | (cocooning effect, | | |
| | Appearance | Noodling | Presence of little | Wait 3 mn after | Absence-Presence |
| | | | flakes/balls/clumps | application, stroke the | |
| | | | on top of the skin | zone where product was | |
| | | | during application | rubbed in and assess | |
| | | | | apparition of little balls | |
| | Texture | Irritation | Sensation of | Assess the tingling | Low-High |
| | | | tingling, irritation, | sensation after applying | |
| | | | tickling or | the product to the skin. | |
| | | | discomfort on the | | |
| | | | skin after | | |
| | | | application of the | | |
| | | | product | | |
| | Odor | Overall | Evaluate the overall | Bring forearm close to | Low-High |
| | | Fragrance | fragrance intensity | the nose and evaluate | |
| | | Intensity | left on skin after | fragrance intensity | |
| | | after | application | | |
| | | application | | | |

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### EXEMPLARY ASPECTS

The following exemplary aspects are provided, the numbering of which is not to be construed as designating levels of importance:

Aspect 1 provides a cosmetic composition comprising:
water;
an emulsifier; and
a thickener
an active mixture, comprising:
   kombucha;
   a mixture of Thermus thermophillus ferment and glycerin
   α-glucan oligosaccharide,
wherein
   a wt:wt ratio of the emulsifier to the thickener are in a range of from about 5:1 to about 1.5:1,
   a minimum stability of the cosmetic composition is measured at 8 weeks in a range of from about 30° C. to about 50° C.,
   a pH of the cosmetic composition measured at a time of manufacturing and at 2 months following manufacturing is in a range of from about 5 to about 6, and
      a viscosity of the cosmetic composition 24 hours after manufacture and at 2 months following manufacturing is in a range of from about 30,000 cps to about 80,000 cps.

Aspect 2 provides the cosmetic composition of Aspect 1, wherein the wt:wt ratio of the of the emulsifier to the thickener are in a range of from about 5:4 to about 5:2.

Aspect 3 provides the cosmetic composition of any one of Aspects 1 or 2, wherein the cosmetic composition is an oil-in-water emulsion.

Aspect 4 provides the cosmetic composition of any one of Aspects 1-3, wherein the emulsifier comprises PEG-40 stearate and the thickener comprises a mixture of sodium acrylates copolymer and lecithin.

Aspect 5 provides the cosmetic composition of any one of Aspects 3 or 4, wherein the thickener is present in the water phase of the cosmetic composition.

Aspect 6 provides the cosmetic composition of any one of Aspects 3-5, wherein the emulsifier is present in the oil phase of the cosmetic composition.

Aspect 7 provides the cosmetic composition of any one of Aspects 3-6, wherein the active mixture is present in a water phase of the cosmetic composition.

Aspect 8 provides the cosmetic composition of any one of Aspects 3-7, wherein the oil phase further comprises:
shea butter;
pentaerythrityl tetraethylhexanoate;
triglyceride;
behenyl alcohol;
glyceryl stearate; or
a mixture thereof.

Aspect 9 provides the cosmetic composition of any one of Aspects 3-8, wherein the water phase comprises:
glycerine;
xanthan gum; or
a mixture thereof.

Aspect 10 provides the cosmetic composition of any one of Aspects 8 or 9, wherein any one or more of:
shea butter is in a range of from about 1 wt % to about 10 wt % of the cosmetic composition;
pentaerythrityl tetraethylhexanoateis in a range of from about 1 wt % to about 8 wt % of the cosmetic composition;
triglyceride is in a range of from about 1 wt % to about 10 wt % of the cosmetic composition;
behenyl alcohol is in a range of from about 1 wt % to about 5 wt % of the cosmetic composition;
glyceryl stearate is in a range of from about 1 wt % to about 5 wt % of the cosmetic composition;
glycerine is in a range of from about 1 wt % to about 10 wt % of the cosmetic composition; or
xanthan gum is in a range of from about 0.1 wt % to about 5 wt % of the cosmetic composition.

Aspect 11 provides the cosmetic composition of any one of Aspects 8-10, wherein any one or more of:
shea butter is in a range of from about 3 wt % to about 7 wt % of the cosmetic composition;
pentaerythrityl tetraethylhexanoateis in a range of from about 2 wt % to about 5 wt % of the cosmetic composition;
triglyceride is in a range of from about 2% to about 8 wt % of the cosmetic composition;
behenyl alcohol is in a range of from about 2 wt % to about 4 wt % of the cosmetic composition;
glyceryl stearate is in a range of from about 2 wt % to about 4 wt % of the cosmetic composition;
glycerine is in a range of from about 3 wt % to about 7 wt % of the cosmetic composition; or
xanthan gum is in a range of from about 0.2 wt % to about 0.7 wt % of the cosmetic composition.

Aspect 12 provides the cosmetic composition of any one of Aspects 1-11, wherein the active mixture is in a range of from about 1 wt % to about 15 wt % of the cosmetic composition.

Aspect 13 provides the cosmetic composition of any one of Aspects 1-12, wherein the active mixture is in a range of from about 5 wt % to about 10 wt % of the cosmetic composition.

Aspect 14 provides the cosmetic composition of any one of Aspects 1-13, wherein
kombucha is in a range of from about 1 wt % to about 5 wt % of the cosmetic composition;
the mixture of Thermus thermophillus ferment and glycerin is in a range of from about 1 wt % to about 10 wt % of the cosmetic composition; and
α-glucan oligosaccharide is in a range of from about 0.1 wt % to about 5 wt % of the cosmetic composition.

Aspect 15 provides the cosmetic composition of any one of Aspects 1-14, wherein
kombucha is in a range of from about 2 wt % to about 4 wt % of the cosmetic composition;
the mixture of Thermus thermophillus ferment and glycerin is in a range of from about 3 wt % to about 7 wt % of the cosmetic composition; and
α-glucan oligosaccharide is in a range of from about 0.5 wt % to about 3 wt % of the cosmetic composition.

Aspect 16 provides the cosmetic composition of any one of Aspects 1-15, wherein the cosmetic composition is a topical skin composition.

Aspect 17 provides a method of making the cosmetic composition of any one of Aspects 1-16, the method comprising:
mixing the water, emulsifier, thickener, and active mixture to form the cosmetic composition.

Aspect 18 provides a method of moisturizing skin, the method comprising topically applying the cosmetic composition of any one of Aspects 1-17 to a person's skin, wherein the cosmetic composition moisturizes the skin.

1. A cosmetic composition comprising:
water;
an emulsifier; and
a thickener
an active mixture, comprising:
kombucha;
a mixture of Thermus thermophillus ferment and glycerin
α-glucan oligosaccharide,
wherein
a wt:wt ratio of the emulsifier to the thickener are in a range of from about 5:4 to about 1.5:2,
a minimum stability of the cosmetic composition is measured at 8 weeks in a range of from about 30° C. to about 50° C.,
a pH of the cosmetic composition measured at a time of manufacturing and at 2 months following manufacturing is in a range of from about 5 to about 6, and
a viscosity of the cosmetic composition 24 hours after manufacture and at 2 months following manufacturing is in a range of from about 30,000 cps to about 80,000 cps as measured with a Brookfield RVT E/20 at a temperature of 25° C.

2. The cosmetic composition according to claim 1, wherein the cosmetic composition is an oil-in-water emulsion.

3. The cosmetic composition of claim 1, wherein the emulsifier comprises PEG-40 stearate and the thickener comprises a mixture of sodium acrylates copolymer and lecithin.

4. The cosmetic composition of claim 2, wherein the thickener is present in the water phase of the cosmetic composition.

5. The cosmetic composition of claim 2, wherein the emulsifier is present in the oil phase of the cosmetic composition.

6. The cosmetic composition of claim 1, wherein the active mixture is present in a water phase of the cosmetic composition.

7. The cosmetic composition of claim 1, wherein the oil phase further comprises:
shea butter;
pentaerythrityl tetraethylhexanoate;
triglyceride;
behenyl alcohol;
glyceryl stearate; or
a mixture thereof.

8. The cosmetic composition of claim 1, wherein the water phase comprises:
glycerine;
xanthan gum; or
a mixture thereof.

9. The cosmetic composition of claim 1, wherein any one or more of:
shea butter is in a range of from about 1 wt % to about 10 wt % of the cosmetic composition;
pentaerythrityl tetraethylhexanoateis in a range of from about 1 wt % to about 8 wt % of the cosmetic composition;
triglyceride is in a range of from about 1 wt % to about 10 wt % of the cosmetic composition;
behenyl alcohol is in a range of from about 1 wt % to about 5 wt % of the cosmetic composition;
glyceryl stearate is in a range of from about 1 wt % to about 5 wt % of the cosmetic composition;
glycerine is in a range of from about 1 wt % to about 10 wt % of the cosmetic composition; or
xanthan gum is in a range of from about 0.1 wt % to about 5 wt % of the cosmetic composition.

10. The cosmetic composition of claim 1, wherein any one or more of:
shea butter is in a range of from about 3 wt % to about 7 wt % of the cosmetic composition;
pentaerythrityl tetraethylhexanoateis in a range of from about 2 wt % to about 5 wt % of the cosmetic composition;
triglyceride is in a range of from about 2% to about 8 wt % of the cosmetic composition;
behenyl alcohol is in a range of from about 2 wt % to about 4 wt % of the cosmetic composition;
glyceryl stearate is in a range of from about 2 wt % to about 4 wt % of the cosmetic composition;
glycerine is in a range of from about 3 wt % to about 7 wt % of the cosmetic composition; or
xanthan gum is in a range of from about 0.2 wt % to about 0.7 wt % of the cosmetic composition.

11. The cosmetic composition of claim 1, wherein the active mixture is in a range of from about 1 wt % to about 15 wt % of the cosmetic composition.

12. The cosmetic composition of claim 1, wherein the active mixture is in a range of from about 5 wt % to about 10 wt % of the cosmetic composition.

13. The cosmetic composition of claim 1, wherein
kombucha is in a range of from about 1 wt % to about 5 wt % of the cosmetic composition;
the mixture of Thermus thermophillus ferment and glycerin is in a range of from about 1 wt % to about 10 wt % of the cosmetic composition; and
α-glucan oligosaccharide is in a range of from about 0.1 wt % to about 5 wt % of the cosmetic composition.

14. The cosmetic composition of claim 1, wherein
kombucha is in a range of from about 2 wt % to about 4 wt % of the cosmetic composition;
the mixture of Thermus thermophillus ferment and glycerin is in a range of from about 3 wt % to about 7 wt % of the cosmetic composition; and
α-glucan oligosaccharide is in a range of from about 0.5 wt % to about 3 wt % of the cosmetic composition.

15. The cosmetic composition of claim 1, wherein the cosmetic composition is a topical skin composition.

16. A method of making the cosmetic composition claim 1, the method comprising:
mixing the water, emulsifier, thickener, and active mixture to form the cosmetic composition.

17. A method of moisturizing skin, the method comprising topically applying the cosmetic composition of claim 1, to a person's skin, wherein the cosmetic composition moisturizes the skin.
